(19) United States
(12) Patent Application Publication
(10) Pub. No.: US 2009/0191258 A1
(43) Pub. Date: Jul. 30, 2009

(54) IODINE-CONTAINING COMPOSITIONS

(76) Inventor: Roy J. Mankovitz, Montecito, CA (US)

Correspondence Address: GIFFORD, KRASS, SPRINKLE,ANDERSON & CITKOWSKI, P.C, PO BOX 7021, TROY, MI, 48007-7021, US

(21) Appl. No.: 12/359,681

(22) Filed: Jan. 26, 2009

Related U.S. Application Data
(60) Provisional application No. 61/023,192, filed on Jan. 24, 2008.

(51) Int. Cl. A61K 33/18; A61L 15/16; A61F 13/15

(52) U.S. Cl. 424/447; 424/669; 424/670; 424/671; 604/304

(57) ABSTRACT

Compositions provided according to embodiments of the present invention include an inorganic clay admixed with an iodine-containing substance. Suitable iodine-containing substances include I₂ and/or one or more iodine salts. Methods of treating a condition or disorder are provided according to the present invention which include administration of a therapeutically effective amount of a composition including an inorganic clay admixed with an iodine-containing substance to an affected subject. Optionally, the composition is administered in an article of manufacture to the affected area.

### REFERENCE TO RELATED APPLICATION

This application claims priority from U.S. Provisional Patent Application Ser. No. 61/023,192, filed Jan. 24, 2008, the entire content of which is incorporated herein by reference.

### FIELD OF THE INVENTION

The present invention relates generally to iodine-containing compositions are provided according to the present invention. Specifically, aspects of the present invention relate to admixtures of clay and inorganic iodine.

### BACKGROUND OF THE INVENTION

By adopting good eating habits, particularly as described in the book R. Mankovitz, The Wellness Project: A Rocket Scientist's Blueprint for Health, Montecito Wellness LLC, 2008, ISBN-13: 978-0980158458 which is hereby incorporated by reference in its entirety, an individual takes a giant step to stop ingesting natural toxins at every meal, thus relieving a burden on the systems used by the body to detoxify.

However, even if the perfect diet is eaten, the accumulated toxic load of a lifetime can prevent nutrient assimilation and can also block and disrupt critical processes throughout the body, causing a great deal of illness.

There is a continuing need for methods and compositions promote health, inhibit microbes and reduce toxins in a subject.

### SUMMARY OF THE INVENTION

A composition provided according to embodiments of the present invention includes an inorganic clay admixed with an iodine-containing substance. Suitable iodine-containing substances include I₂ and/or one or more iodine salts.

In preferred embodiments, the inorganic clay included is unmodified natural clay. In a preferred embodiment, the inorganic clay is not modified to include quaternary amines.

Iodine salts that can be included in a composition according to embodiments of the present invention are KI, NaI, LiI, CaI₂ and MgI₂. Two or more of KI, NaI, LiI, CaI₂ and MgI₂, are admixed with the clay in particular embodiments of the present invention.

The clay included in a composition according to embodiments of the present invention can be hydrated, partially hydrated or substantially water-free.

Optionally, a clay included in a composition according to embodiments of the present invention is a calcium bentonite and/or a sodium bentonite.

Articles of manufacture according to embodiments of the present invention include a composition including an inorganic clay admixed with an iodine-containing substance, the composition in contact with a substrate. Examples of inventive articles of manufacture include poultices, bandages, wound dressings, diapers, adult incontinence pads, dental tampons, medical tampons, surgical tampons, nasal tampons, and feminine hygiene products such as catamenial tampons, sanitary napkins and panty liners

Methods of treating urogenital itching and irritation are provided according to the present invention which include application of a therapeutically effective amount of a composition including an inorganic clay admixed with an iodine-containing substance to an affected area. Optionally, the composition is administered in an article of manufacture to the affected area.

Methods of promoting health in an individual are provided according to embodiments of the present invention which include administering to an individual a composition comprising an inorganic clay admixed with an iodine-containing substance selected from the group consisting of: I₂, an iodine salt, and a mixture thereof.

### BRIEF DESCRIPTION OF THE DRAWINGS

FIG. 1 is a schematic illustration of an article of manufacture according to embodiments of the present invention including a composition described herein in contact with a substrate and an optional top sheet;

FIG. 2 is a schematic illustration of an article of manufacture according to embodiments of the present invention including a composition described herein contained within a substrate and an optional top sheet; and

FIG. 3 is a schematic illustration of an article of manufacture according to embodiments of the present invention for insertion or partial insertion into the body of a subject.

### DETAILED DESCRIPTION OF THE INVENTION

Preferred embodiments of compositions of the present invention include iodine admixed with clay. These compositions are administered to a subject to promote health, inhibit microbes and to reduce toxins in contact with the subject.

A composition provided according to embodiments of the present invention includes an inorganic clay admixed with an iodine-containing substance. Suitable iodine-containing substances include I₂ and/or one or more iodine salts.

In preferred embodiments, the inorganic clay included is unmodified natural clay. In a preferred embodiment, the inorganic clay is not modified to include quaternary amines.

Iodine admixed with clay is inorganic iodine and/or an inorganic iodine salt. Iodine salts that can be included in a composition according to embodiments of the present invention are KI, NaI, LiI, CaI₂ and MgI₂. Two or more of KI, NaI, LiI, CaI₂ and MgI₂, are admixed with the clay in particular embodiments of the present invention. Lugol's iodine, an aqueous mixture of iodine (I₂) and potassium iodide, is admixed with clay in particular embodiments of the present invention.

Clay included in a composition of the present invention is preferably a natural clay. Preferred natural clays are calcium bentonites and sodium bentonites illustratively including, and not limited to, Pascalite, Terramin and Redmond clay. Natural clays are obtained commercially or isolated from the natural environment. Natural clays are well-known in the art, for example, as described in F. Bergaya et al., Eds., Handbook of Clay Science, Elsevier Science, 2006.

In particular embodiments of inventive compositions, inorganic iodine and/or one or more inorganic iodine salts is mixed with hydrated clay. Powdered clay is hydrated by mixing with an aqueous medium to get the desired consistency for the intended use. A preferred aqueous medium is water.

Inorganic iodine and/or an inorganic iodine salt is mixed with the hydrated clay to a desired concentration. In preferred embodiments, iodine is mixed with the hydrated clay to a maximal concentration. The maximal concentration is the highest concentration of iodine admixed with hydrated clay where the iodine does not stain the skin when the composition is applied topically to a subject.

In embodiments of the present invention, the iodine-containing substance, inorganic iodine and/or one or more inorganic iodine salts, and the clay are present in a composition in a ratio of about 0.01:100—about 10:1 on a weight/weight basis.

In further embodiments, a substantially dry composition including iodine and clay is provided according to the present invention. For example, a substantially dry composition is produced by admixing powdered clay and solid iodine. In one example, a substantially dry composition includes less than 0.1% water by weight of the composition.

In a further embodiment, a substantially dry composition is produced by mixing iodine with hydrated clay and desiccating the mixture. Maximal concentration of iodine and/or an iodine salt in a substantially dry composition is that where the iodine and/or an iodine salt remains totally adsorbed and/or absorbed in the clay.

The maximal concentration is different for different clay compositions and is therefore determined experimentally.

Compositions of the present invention are administered to a subject in various forms depending on the intended application. Compositions are illustratively administered in the form of poultices, bandages, toothpaste, mouthwash/gargle, soap, shampoo, cosmetic base for troubled skin, and as slurrys or solids for internal consumption. Formulation of compositions of the present invention includes adjustment of the composition to have a desired consistency for a particular application. For example, a thicker, more gel-like composition is desirable for a poultice whereas a relatively thin, more water-like consistency is desirable for a mouthwash.

In a particular embodiment of a substantially dry composition is administered contained in an ingestible capsule for oral ingestion. Capsule formulation is optionally designed to target release of the contained composition of the present invention to a particular target region, such as the stomach, small intestine and/or or large intestine.

Detailed information concerning customary excipients, equipment and processes for preparing dosage forms is found in Pharmaceutical Dosage Forms: Tablets, eds. H. A. Lieberman et al., New York: Marcel Dekker, Inc., 1989; and in L. V. Allen, Jr. et al., Ansel's Pharmaceutical Dosage Forms and Drug Delivery Systems, 8th Ed., Philadelphia, Pa.: Lippincott, Williams & Wilkins, 2004; A. R. Gennaro, Remington: The Science and Practice of Pharmacy, Lippincott Williams & Wilkins, 20th ed., 2003; and J. G. Hardman et al., Goodman & Gilman's The Pharmacological Basis of Therapeutics, McGraw-Hill Professional, 10th ed., 2001.

Compositions of the present invention are administered to human subjects and non-human subjects.

Methods of treating a condition or disorder are provided according to the present invention which include administration of a therapeutically effective amount of a composition including an inorganic clay admixed with an iodine-containing substance to an affected subject. Optionally, the composition is administered in an article of manufacture to the affected area.

A therapeutically effective amount of a composition of the present invention is an amount effective to ameliorate the condition or disorder, such as by amelioration of a symptom or sign of the condition or disorder.

A therapeutically effective amount administered in one or more doses depends on various factors such as the age of the subject, the severity and type of condition being treated in the subject, the general condition of the subject, the route and form of administration of the composition being administered and the particular composition administered. One of ordinary skill in the art will be able to ascertain a therapeutically effective amount without undue experimentation in view of the present disclosure and what is known in the art.

Methods of treating urogenital itching and irritation are provided according to the present invention which include application of a therapeutically effective amount of a composition including an inorganic clay admixed with an iodine-containing substance to an affected area. Optionally, the composition is administered in an article of manufacture to the affected area.

Bodily fluids including, but not limited to, sweat, wound exudates, urine, mucous and vaginal fluids can irritate skin if not removed from skin or allowed to evaporate. Bodily fluids can support the growth of microbes and chemical content of the fluids can also cause skin irritation. There is a continuing need for methods and articles for absorption and disinfection of bodily fluids.

Articles of manufacture according to the present invention are configured to absorb and disinfect bodily fluids. Poultices, bandages, wound dressings, diapers, adult incontinence pads, dental tampons, medical tampons, surgical tampons, nasal tampons, and feminine hygiene products such as catamenial tampons, sanitary napkins and panty liners are examples of articles of manufacture according to embodiments of the present invention.

In particular embodiments, articles of manufacture are configured to absorb and disinfect bodily fluids include a support in contact with a composition of the present invention. In operation, the article is placed in contact with the body of a user so that one or more bodily fluids is absorbed and disinfected by the composition on the support. Depending on the particular configuration, the support can be permeable or impermeable to bodily fluids. Optionally, one or more portions of the support can be permeable or impermeable to bodily fluids. For example, a portion of the support positioned away from the body of the user when the article is in use, such a surface in contact with user clothing, is impermeable to bodily fluids. In a further example, a portion of the support positioned toward the body of the user when the article is in use, is permeable to bodily fluids.

In further embodiments, a top sheet covers the composition and, in operation, the top sheet is in contact with the body of a user and one or more bodily fluids flows through the top sheet and is absorbed and disinfected by the composition on the support.

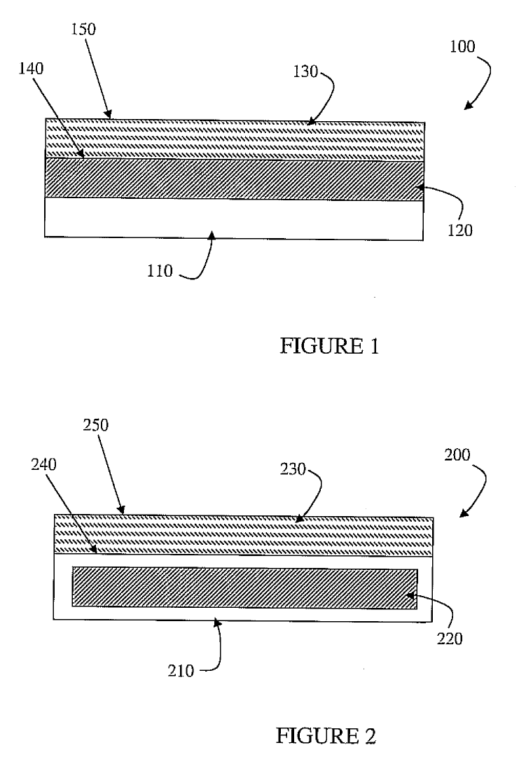

FIG. 1 is a schematic illustration of a cross-sectional view of an article of the present invention 100, including a support 110 in contact with a composition of the present invention 120, and an optional top sheet 130. Surfaces 140 and 150 are positioned towards the body of a user when the article is in use.

A composition of the present invention in contact with a support is optionally contained within the support which is permeable to bodily fluids and impermeable to the composition. FIG. 2 is a schematic illustration of an article of the present invention 200, including a support 210 configured to contain a composition of the present invention 220, and an optional top sheet 230. Surfaces 240 and 250 are positioned towards the body of a user when the article is in use.

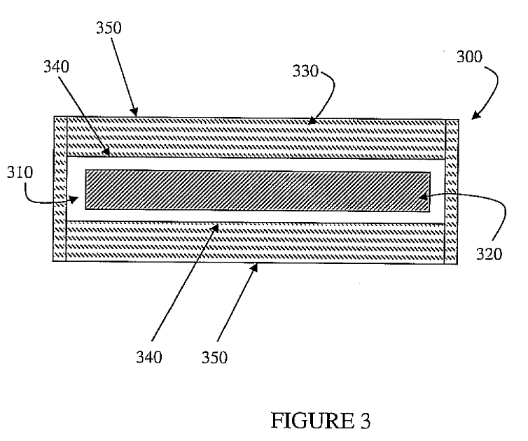

In a further embodiment, an article of the present invention is configured to be inserted into the body of a user or partially inserted into the body of a user. FIG. 3 is a schematic illustration of an article of the present invention 300, including a support 310 configured to contain a composition of the present invention 320, and an optional top sheet 330. Surfaces 340 and 350 are positioned towards the body of a user when the article is in use. A tampon is an example of an embodiment in which an inventive article is inserted into a user's body.

In preferred embodiments, a composition of the present invention included in an article of manufacture for absorbing and disinfecting bodily fluids is a substantially dry composition as described herein. Contact between the substantially dry composition and bodily fluids results in activation of the composition by hydration and the bodily fluids are absorbed and disinfected by the composition.

Articles according to the present invention are manufactured from a wide variety of materials including woven and non-woven materials and/or polymeric materials. Woven and non-woven materials can include natural fibers, such as cotton, hemp or linen, and synthetic fibers such as polymeric fibers illustratively including polyester, polyethylene and/or polypropylene fibers. Combinations of natural and synthetic fibers can be used. Polymeric materials used can include thermoplastic films, such as polyethylene or polypropylene films. Optionally, a thermoplastic film is coated with, or otherwise adhered to a woven or non-woven material.

Methods of treating urogential itching and irritation in an infant, child or adult subject are provided according to embodiments of the present invention which include application of a composition of the present invention to an affected area. The composition is optionally applied using an appropriate article of manufacture, such as a diaper, adult incontinence pad, and/or feminine hygiene product.

Any patents or publications mentioned in this specification are incorporated herein by reference to the same extent as if each individual publication is specifically and individually indicated to be incorporated by reference.

The compositions, article and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1. A composition, comprising:
an inorganic clay admixed with an iodine-containing substance selected from the group consisting of: I₂, an iodine salt, and a mixture thereof.

2. The composition of claim 1, wherein the inorganic clay is unmodified natural clay.

3. The composition of claim 1, wherein the iodine salt is selected from the group consisting of: KI, NaI, LiI, CaI₂ and MgI₂.

4. The composition of claim 1, wherein two or more iodine salts selected from the group consisting of: KI, NaI, LiI, CaI₂ and MgI₂, are admixed with the clay.

5. The composition of claim 1, wherein the iodine-containing substance is Lugol's iodine.

6. The composition of claim 1, wherein the clay is hydrated.

7. The composition of claim 1, wherein the clay is substantially water-free.

8. The composition of claim 1, comprising less than 0.1% water by weight of the composition.

9. The composition of claim 1, wherein the clay is a calcium bentonite.

10. The composition of claim 1, wherein the clay is a sodium bentonite.

11. The composition of claim 1, wherein the clay is selected from the group consisting of: Pascalite, Terramin and Redmond clay.

12. An article of manufacture, comprising:
the composition of claim 1 in contact with a substrate.

13. The article of manufacture of claim 12, wherein the article of manufacture is selected from the group consisting of: a poultice, a bandage, a diaper, a wound dressing, an adult incontinence pad, a dental tampon, a medical tampon, a surgical tampon, a nasal tampon, and a feminine hygiene product.

14. The article of manufacture of claim 13, wherein the feminine hygiene product is selected from the group consisting of: a catamenial tampon, a sanitary napkin and a panty liner.

15. A method of promoting health, comprising:
administering to an individual a composition comprising an inorganic clay admixed with an iodine-containing substance selected from the group consisting of: I₂, an iodine salt, and a mixture thereof.
